(19) United States
(12) Patent Application Publication
(10) Pub. No.: US 2006/0276701 A1
(43) Pub. Date: Dec. 7, 2006

(54) Detection and stimulus conditioning system for sleep apnea

(76) Inventor: Charles D. Ray, Santa Barbara, CA (US)

Correspondence Address: DICKE, BILLIG & CZAJA, P.L.L.C., FIFTH STREET TOWERS, 100 SOUTH FIFTH STREET, SUITE 2250, MINNEAPOLIS, MN, 55402, US

(21) Appl. No.: 11/144,446

(22) Filed: Jun. 3, 2005

(51) Int. Cl. A61B 5/05; A61B 5/00

(52) U.S. Cl. 600/354; 600/300

(57) ABSTRACT

An autonomic conditioning device for treatment of sleep apnea is disclosed. The device includes a control unit and a user mounting device carrying at least one respiratory sensor, at least one conditioning signal electrode, and at least one punitive signal electrode that are electrically connected to the control unit. In this regard, during use the respiratory sensor(s) sense information indicative of wearer respiration, and the control unit is adapted to prompt the conditioning signal electrode(s) to transmit a warning to the wearer upon detecting a variation in respiratory cycle time. The control unit is further adapted to prompt the punitive signal electrode(s) to transmit a painful pulse to the wearer upon identifying an unheeded warning from the conditioning signal electrode.

### FIELD OF THE INVENTION

Aspects of the present invention include a stimulus conditioning system for treatment of sleep apnea, and more particularly, an autonomic conditioning system for treatment of central apnea.

### BACKGROUND

Apnea, (Greek for no breath) is a cessation of breathing or periodic breathing. When the period of non-breathing is greater than 30 seconds between breaths it is referred to as a period of apnea, however in practice respiratory gaps of more than 5 to 7 seconds may indicate clinical apnea. Nonetheless, respiratory periodicity varies normally and is slower during rest or sleep. When the in and out respirations continue without pause and are rapid, this too is abnormal, being referred to as hyperpnea or hyperventilation. Normally, the breathing rate to a large extent determines the content of oxygen and carbon dioxide, the respiratory gases, in the blood and tissues. A timing/chemical sensing organ in the floor of the brain stem called the respiratory pacemaker, sets the normal breathing rate depending on the concentrations of these gases, plus variations in the acidity of the blood and a wide variety of other potential influences, such as, drugs, fatigue, alcohol and brain or heart abnormalities. Periods of apnea in which breathing ceases for a few seconds before resuming normally occur during sleep. When longer periods of apnea alternate with periods of ‘catch up’ rapid, heavy breathing, such irregular respiratory cycles are called Cheyne-Stokes respiration. Cheyne-Stokes respiration may be indicative of any of the above influences plus strokes, degenerative diseases of the nervous system such as Parkinson's syndrome, brain stem injury or abnormalities such as tumors or even congenital disorders of brain stem cells or of the heart.

The intermittent respiration in sleep apnea results in blood oxygen desaturation, brain arousal patterns and changes in heart rate. In a typical sleep study of apneas, 78% of the episodes resulted in some blood oxygen desaturation, 63% in partial to complete arousal, and 73% in heart rate increase. Long term, these effects can contribute to dementia, cardiac irregularities, high blood pressure and weakened heart muscles. Importantly, apnea may cause these conditions as well as result from them. Sleep apnea is potentially lethal, particularly when some associated disease is present. Many cases of sudden infant death syndrome (SIDS) are due to prolonged apnea. Apnea resulting in poor sleep due to arousal wakefulness when catching up to correct abnormal respiratory gas concentrations, often results in sleepiness during the following day which contributes to inattention to details, day dreaming, poor work habits, auto accidents, inactivity and obesity. Obesity then also contributes to the apnea.

Sleep disorders affect up to 90% of the population, and of these cases, sleep apnea is the most important worldwide problem, occurring in about 3 to 5% of the general population. International medical literature is filled with various studies of causation, diagnostic and treatment means for apnea. It is often a serious and potentially deadly disorder.

There are two principal types of apnea: obstructive and central. Obstructive (by far the most common) is found where sagging muscles of the throat and neck or disorders of the nasal passages cause restrictions in air flow, particularly at night. The other type, central, is much less common and more difficult to diagnose and treat, which arises primarily from disorders of the respiratory pacemaker in the brain stem not sending signals to the respiratory muscles to initiate respirations. This can occur through a wide variety of abnormal influences, including those listed above. Prolonged apnea or respiratory arrest can occur during epileptic attacks (Nelson DA, Ray CD: Respiratory arrest from seizure discharges in the limbic system. Archives of Neurology 19:199-207, 1968.)

There are also cases having a hybrid or mixed form of apnea caused by a mixture of the two main causes, obstructive and central. The treatment of each type is quite different although some form of applied pressure to the inspired air is presently the most common for any of them. Additionally, various devices that improve the position of the neck at night, such as corrective pillows or devices that thrust the jaw forward aid in a better opening of the air passage. Age with associated muscular weakness contributes to throat sag as also does obesity. Direct treatment of this obstructive condition may involve weight loss or even surgery to correct sagging tissues. Obesity as a contributing factor improves through weight reduction. Several types of sleeping medicine may be harmful but not all are. Multiple studies over the last several years have failed to find a suitable medical treatment means for any of the types of apnea.

Particularly when sleeping supine, flat on the back, the harsh rasping flapping of sagging throat tissues usually produces snoring and intermittent snorting while gasping for breath. Of course, snoring in some form is common to all persons under different conditions but is particularly noisy among older, obese males. Any partial obstruction to nasal passages, such as a common cold, flu, swollen tonsils or a deviation, infection or a growth in the nasal passage also contribute to snoring. Obstructive apnea can be helped through throat exercises and sleeping on one's side rather than the back. In suspected or more serious cases, an overnight sleep study in a sleep laboratory is needed to make a full and correct diagnosis of the cause and treatment of apnea.

Doctors usually advise snorers to lose weight, avoid alcohol within two hours before sleep, as well as sleep-inducing tranquilizers, sleeping pills, and sleep-inducing antihistamines before going to bed. When snoring and rapid, heavy breathing are interspersed with seven- to ten-second irregular periods of halting in breathing, the problem may be sleep apnea, often leading to excessive sleepiness during the day.

Treatment of sleep apnea is not simple since there are many different causes of disturbed sleep and disturbed respirations during sleep, as indicated above. The most important diagnostic study is called Polysomnography which records several body functions during sleep, including respiratory patterns, electrical activity of the brain, movement of the eyes, general body and chest muscular activity, heart rate, respiratory effort, air flow, and blood oxygen levels. These diagnostic tests usually are performed in a sleep laboratory although newer recording devices permit studies in the patient's home. The sleep physiologist examines the records and draws correlations between observed phenomena and the stages of sleep searching for diagnostic patterns. The patterns related to true apnea, obstructive or central or mixed, are most usually detectable through these sleep physiology records. At some point during the sleep study the patient is awakened and given a positive pressure demand breathing assist mask and air pump system to use and later in the same session or sometimes during a subsequent sleep study session, the changes and improvements are recorded. For obstructive cases the positive pressure is applied during inspiration when it serves to dilate the air passages and overcome airflow resistances.

Central apneic cases, on the other hand, require positive pressure during all phases of the respiratory cycle although stronger during inhalation. A side effect of the increased pharyngeal and pulmonary pressure is the apparent driving of surface contaminants lying on the respiratory mucous membranes deeper into the membranes giving the patient the feeling of congestions and increased mucus production as though having a bad head cold, an undesirable effect. The breathing assist devices are house current operated, a bit noisy and the air tubing and face mask pose problems of acceptance by patient and bed partner plus a substantial additional bulk when traveling.

Sleep apnea is widely prevalent and potentially dangerous to the patient. Both treating physicians and patients will welcome advances in the treatment of sleep apnea.

### SUMMARY

One aspect of the present invention provides an autonomic conditioning device for treatment of sleep apnea. The device includes a control unit and a user mounting device carrying at least one respiratory sensor, at least one conditioning signal electrode, and at least one punitive signal electrode that are electrically connected to the control unit. In this regard, during use the respiratory sensor(s) sense information indicative of wearer respiration, and the control unit is adapted to prompt the conditioning signal electrode(s) to transmit a warning to the wearer upon detecting a variation in respiratory cycle time. The control unit is further adapted to prompt the punitive signal electrode(s) to transmit a painful pulse to the wearer upon identifying an unheeded warning from the conditioning signal electrode.

Another aspect of the present invention provides a method of autonomic conditioning of a patient in treating sleep apnea. The method includes removably attaching sensor(s) and actuator(s) to a user, the sensor/actuator being coupled to a control unit. A respiration cycle of the patient is sensed. The method additionally includes conditioning the patient to maintain a desired respiration cycle. The method provides for negatively rewarding the patient for a failure to maintain a desired respiration cycle.

In one embodiment, the novel system for diagnosis and treatment of sleep apnea consists of a belt on which is mounted detectors that sense important parameters of respiration, namely: electrographic potentials arising from contraction of respiratory musculature, expansion of the chest and oxygen saturation by transdermal oxymetry. The output of these parameters are fed into a control unit mounted on the same belt having a differentiator unit to determine which of the measured parameters yields the most reliable indicator of respiratory rhythm. This comparator then uses this criterion until it might become less dependable due to body position or other change then the more reliable parameter is then followed for timing of respirations. An additional part of the control unit has adjustable controls for interval timing between the last inspiration and onset of the adjustable warning vibration and the time between the warning (conditioning signal) and the application of the adjustable, small painful shock. These preset times are reset by the onset of a new respiratory cycle. The entire novel system, belt and control unit are battery operated, highly portable and relatively small, quite suitable for travel. The patient's bed partner should perceive neither of the feedback signals.

### BRIEF DESCRIPTION OF THE DRAWINGS

Embodiments of the invention are better understood with reference to the following drawings, where like reference numerals designate corresponding similar parts. The drawings are schematic and not to scale. Both a numbering and lettering scheme is used in designating the various components of the system. Details are generally in three categories: numbered diagrams of device construction, letter labeling of electrode detectors and lettered designation of parts of a representative electronic circuit schematic. Electronic circuit designers familiar with the art here used can provide similar diagrams and circuit designs without departing from the intent and purpose of the here given design.

FIG. 1 is a diagrammatic oblique view of the novel detector or transducer electrode-holding, adjustable user mounting device worn by the patient around the naked chest. All electrodes are in intimate proximity with the bare skin which may require preparation, such as the removal of natural skin oils, to reduce the surface impedance of the electrode-skin junction and therefore detector noise or artifact.

FIG. 2 is a diagrammatic plan view of the belt surrounding the bare chest with the electrodes and detectors mounted thereupon in conjunction with a control unit. The circuit diagram features the use of signal conditioning amplifiers, each having adjustability within stated parameters. Each component detector or conditioning electrode, namely, those that stimulate by vibration or by small painful shock, is appropriately isolated and under control by a master control box that also provides power to the components through self-contained batteries. An optional remote finger oxymetry unit is also shown.

### DETAILED DESCRIPTION

In the following Detailed Description, reference is made to the accompanying drawings which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments of the present invention can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

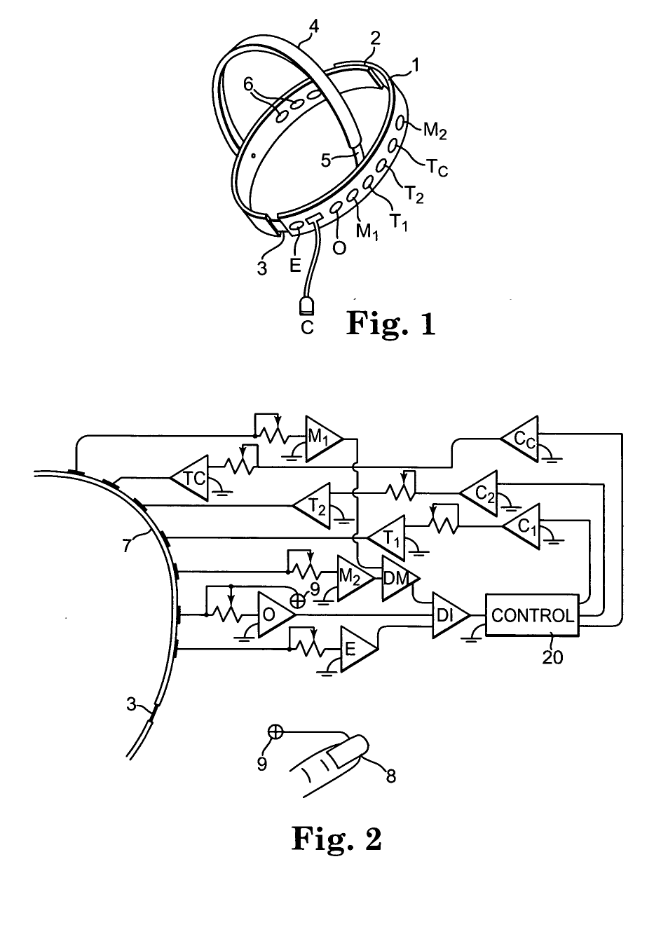

FIG. 1 diagrammatically illustrates an autonomic conditioning device for treatment of sleep apnea according to one embodiment of the present invention. In general terms, the device includes a user mounting device (such as a belt), one or more sensors carried by the mounting device for detecting physical traits of the user indicative of breathing, one or more actuators (e.g., vibrator) for prompting breathing by the user, and a control unit.

One embodiment of a user mounting device is shown in FIG. 1 and includes a thin, inelastic, comfortable, adjustable torso belt 1 adapted to be wrapped around the bare chest of a user (not shown), where the skin has been properly cleansed of surface oil to improve electrode contact. The belt 1 can be tightened around the chest by an overlapping of hook and loop fastener junction 2 in accordance with one embodiment. Additionally, there is, in one embodiment, an elastic joint 3 allowing the distention of the belt 1 with respiratory changes in chest circumference; the elastic joint 3 also contains a transducer E that indicates chest expansion. The position of the belt 1 can be further maintained on the chest by a cross-shoulder strap 4 having an adjustable hook and loop fastener 5.

Respiratory sensors involved in the detection of respiratory activity in accordance with one embodiment are carried by the belt 1 and include: Oxygen saturation of the blood, using reflection transdermal (skin) oxymetry utilizes a suitable transducer designated at ‘O’. Contraction of respiratory muscles is detected by the paired electrodes designated “M₁” and “M₂”, widely spaced on the chest belt 1. Additional detector electrodes 6 may be positioned on the posterior surface of the belt 1 if required for clarity of the signals. In addition, signal conditioning vibrator T₁ is carried by the belt 1, as is a punitive shocking electrode T₂. A common conditioning reference electrode T₂ can also be provided that shares with T₂, so that two points of slight pain are felt although only one with vibration (the conditioning signal). The electrode wires are brought through a protective layer of the belt 1 to a connector C which attaches to a control unit (not shown).

FIG. 2 is a diagrammatic schematic showing various electrodes and transducers (i.e., respiratory sensors) as they typically interface through adjustable signal conditioning amplifiers in contact with the battery operated control unit 20 according to one embodiment of the present invention. The electrodes of the respiratory sensors are in direct, low-impedance contact with the bare skin 7 of the user's chest wall. Each electrode or transducer is connected through its individual adjustable signal conditioning amplifier and designated thusly, organized in this diagram from below upwards to parallel those as shown on the belt 1 of FIG. 1: Chest Expansion detector ‘E,’ ‘O’=oxymeter (transdermal skin reflection/absorption unit) with an optional outboard connector to a finger blood gas oxymeter 8 through a connector 9. M₁ and M₂₌Muscular contraction potentials that pass through a compound differentiator-integrator amplifier DM and then in common with the outputs of E and O amplifiers into an input differential selective amplifier D1 into the CONTROL unit 20. Selectable output from the CONTROL unit 20 pass appropriately into the conditioning amplifiers C₁, C₂ and C_{c} (Common electrode). Output from the control amplifiers pass into the designated output amplifiers, each to its own, T₁, T₂ or T_{c}. The above-described circuitry can be carried on the belt, or portions thereof maintained with a housing associated with the control unit 20. Regardless, the control unit 20 includes a processor adapted to interpret information from the sensors to determine respiration characteristics of the wearer as well as to prompt operation of the vibrator T₁ and the shock electrode T₂ upon recognizing a respiratory concern, as described below.

Method of Use

Training sessions are conducted during waking hours while comfortably wearing the belt. The patient holds his breath in either inspiration or expiration and awaits the adjustable warning vibrator signal. Then on continuing to hold his breath he receives the localized, adjustable slightly painful negatively rewarding stimulus. Such experiences give the patient the appreciation of the detectors governing the timing of signal feedback as well as the opportunity to experience both the positive and the negative rewards, adjusting them to suit his needs, meanwhile additionally assisting in the preparation for later conditioning of his natural automatic, neurophysiologic respiratory pacemaker. Further, the patient is less apprehensive about the intensity of the feedback signals if and when they occur during sleep. On full conditioning, the patient will automatically not allow respirations to pause beyond the warning vibration with the anticipated result that the natural act of respiration will become more automatically conditioned. Later, the patient can disable the stimulus electronics of the device and see if his respirations then fall within the permitted cycle time, leading to his weaning entirely from the system. At any time of uncertainty or subsequent need the patient may resume the use of the system.

The electronic circuitry compares the outputs of the input detectors, respiratory expansion, electromuscular potentials and blood oxygen saturation and determines which of these parameters more reliably indicates the presence of respiratory action automatically setting that parameter as the basis of measurement of respiratory interval. The system can change the reference input signal as indicated by signal noise or poor responsiveness on the part of any of the electrode detectors. This determined interval regulates the time delay settings for the conditioning vibration and punitive shock, although each has a range that is manually adjustable or preset. Multiple shocks can also be preset to respond to dangerously prolonged apneic periods, beyond the preset intervals. This determination of interval is under control by the CONTROL unit utilizing the input information from the detector electrodes mounted on the belt and in contact with the bare skin. Should no input signal be detected, indicating a prolonged period of apnea, the output signals for conditioning and multiple shocks follow normally, preventing a potentially lethal apneic state. When not in use, the system is removed and the CONTROL unit turned off.

Example of Use

The novel device can be used by the following method. A patient suspected of suffering from central or combined sleep apnea, or one found to have either condition by prior sleep study, can be fitted with the system disclosed here either in a sleep laboratory or at home and the quality of sleep determined. The system should undergo trial training during waking hours to make the patient aware of the conditioning and punitive signals with breath holding. He should also become aware of the method for skin preparation (which may require shaving of dense hairs), belt application and comfortable adjustment as well as the determination of clear signals from the input transducers. Appropriate adjustments should become routine to him. The control unit will not function optimally if input conditions are inappropriate, and the patient should learn the signs of same. There being a direct correlation between apnea and deterioration of sleep quality/quantity, the patient will, on awakening the following day, immediately realize the effect of the novel device on his sleep quantity/quality. If there is substantial doubt regarding the fitting and operation or clinical results of the use of the device system, a suitably trained sleep therapist should be available to make appropriate adjustments or corrections. The patient and his bed partner should be aware of improper operation and undesirable outcomes and be prepared to describe them in detail to the therapist. Both safety and effectiveness of the system are paramount to its continued use.

Advantages

The novel system is used in both treating and curing uncomplicated central or combined sleep apnea, for which there is presently no reasonable treatment and no know cure. In that there are several potential causes of central apnea, some of which are not reversible, such as following a stroke or arising from a brain stem tumor or a degenerative disease such as Parkinson's, the novel device may potentially be useful in treating such cases, depending on the nature and extent of the precipitating pathology, but certainly not in curing the problem. Presently used treatment for these conditions of apnea consists primarily of the application of high positive pressure breathing assist pumps applying inspiratory pressures and then maintaining a level of pressure between inspiratory phases to have some persistence in inflating the lungs. This method is uncomfortable, bulky, and rather noisy and has undesirable effects on the nasal, pharyngeal and respiratory mucous membranes often producing head cold-like symptoms, sinus pain and congestion. It appears that the continued high pressures drive surface contaminants deeper into the mucosa leading to a chronic irritation. These pumps operate on home current and do not travel well.

The novel system is compact, comfortable to wear and silent. It is battery operated and its presence and operation are undetected by the wearer's bed partner. The control unit should be constructed as small and thin as possible so as not to interfere with sleep activities and comforts.

While the preferred embodiments of the invention have been described, it should be understood that various changes, adaptations and modifications may be made therein by those skilled in the art without departing from the spirit of the invention and the scope of the appended claims.

The novel system disclosed here relates to the treatment of the potentially dangerous and even lethal condition of irregular, intermittent or cessation of breathing during sleep, known as sleep apnea. This condition is a common problem worldwide and there are no know medical treatments available at this time. Of the two types of sleep apnea, namely obstructive and central, the former is causes by the sagging of tissues along the respiratory pathway and the latter by an abnormality affecting the respiratory pacemaker center in the brain stem causing it to fail in sending signals to respiratory muscles to begin respiratory efforts.

Obstructive apnea is treated by a number of successful means, like losing weight, exercises and surgery to the throat or the use of an intermittent positive pressure breath-assisting device.

Central apnea, the more dangerous, has no simple or highly successful treatment although continuous positive pressure respiratory assistance or the implantation of a complex phrenic (respiratory) nerve pacemaker/stimulator can be used. These devices are complex, difficult to use, require considerable maintenance, are expensive and are known to be less than fully reliable.

The novel system disclosed here utilizes a completely different and novel approach where a form of autonomic (automatic nervous system) classical conditioning is used to train the respiratory pacemaker to resume its normal functional control. Detectors of respiration are worn as a belt by the patient and respiration is detected using a variety of important parametric transducers. When the apneic cycle is longer than a preset time, varying from 5 to 15 seconds, a warning (conditioning) vibratory signal is sent to the patient via an output transducer mounted on the belt. This may awaken the patient at first but later becomes an unconscious stimulation acting as the conditioning signal. If a respiratory cycle still does not begin within a preset time, varying from 5 to 15 seconds, a small but painful shock is sent via another output transducer mounted on the same belt, to the patient's skin. This shock will arouse the patient causing him to breathe, which then restarts the respiratory cycle. In accord with other known autonomic conditioning studies, this method will reestablish a normal respiratory rhythm and also prevent prolonged respiratory arrest that could be lethal.

In terms of classical conditioning the positive feedback is the onset of breathing and the back-up negative or punishing signal (the mildly painful shock to the skin of the chest wall) provides a negative reward for not breathing. The combination of these two signals, from this classical point of view provides the strongest stimulus, namely, positive and negative rewards for respondent action, is most likely to accelerate operant conditioning or learning. Autonomic conditioning has been applied to highly automatic bodily functions, such as the response of pupil diameter to light, as demonstrated much earlier by my own mentor in graduate school (Cason H: The conditioned pupillary reflex. J. ExpPsychol 5:108-46, April, 1922.)

Further work has shown that the circulation in such as a single kidney can be bought under conditioning control in animal experiments. The system is therefore a novel combined respiratory monitor and apnea treatment means through the application of positive and negative operant conditioning of an automatic control mechanism that has undergone undesirable changes. The novel device system is worn during daytime training cycles as well as at night. It is anticipated that in many patients the method will reestablish normal functions and will uniquely extinguish its own need which breathing assist devices cannot do. Breathing assist devices provide a back up to the abnormal changes in the respiratory pacemaker but offer no training to change the improper response.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific embodiments discussed herein. Therefore, it is intended that this invention be limited only by the claims and the equivalents thereof.

What is claimed is:

1. An autonomic conditioning device for treatment of sleep apnea comprising:
user mounting device comprising:
at least one respiratory sensor, at least one conditioning signal electrode,
at least one punitive signal electrode; and
a control unit electrically connected to each of the sensor and electrodes;
wherein during use, the user mounting device is in contact with skin of a wearer and the at least one respiratory sensor senses information indicative of wearer respiration, and further wherein the control unit is adapted to prompt the at least one conditioning signal electrode to transmit a warning to the wearer upon detecting a variation in respiratory cycle time and prompt the at least one punitive signal electrode to transmit a painful pulse to the wearer upon recognizing an unheeded warning.

2. The autonomic conditioning device of claim 1, wherein the mounting device defines an interior surface, an exterior surface, a posterior section, and an anterior section, and further wherein the at least one respiratory sensor is disposed on the interior surface of the anterior section.

3. The autonomic conditioning device of claim 1, wherein the at least one respiratory sensor includes at least one of a chest expansion transducer, a response electrode, a transdermal oxygen sensor, and a muscle contraction sensor.

4. The autonomic conditioning device of claim 1, wherein the mounting device is an adjustable torso belt that defines an elastic joint, and further wherein at least one chest expansion transducer is coupled to the elastic joint.

5. The autonomic conditioning device of claim 1, wherein the mounting device is an adjustable torso belt that includes a hook and loop fastening system.

6. The autonomic conditioning device of claim 1, further comprising:
a common conditioning reference electrode disposed on the mounting device a distance from the at least one punitive signal electrode.

7. The autonomic conditioning device of claim 1, wherein the control unit is a battery operated control unit.

8. The autonomic conditioning device of claim 1, wherein the control unit includes an adjustable signal conditioning amplifier for each of the at least one respiratory sensor, conditioning signal electrode, and punitive signal electrode.

9. The autonomic conditioning device of claim 1, further comprising:
a finger blood gas oxymeter coupled to the control unit.

10. The autonomic conditioning device of claim 1, wherein the control unit includes means to automatically sense and set an interval of operation from a last wearer inspiration until onset of the warning to the wearer, and automatically sense and set an interval between the warning to the wearer and onset of the painful pulse to the wearer.

11. The autonomic conditioning device of claim 10, wherein the control unit includes means for the wearer to manually override the automatically sensed and set intervals.

12. The autonomic conditioning device of claim 1, wherein the control unit is adapted to determine reliable indicators of respiratory interval timing from among a plurality of sensed respiratory parameters.

13. The autonomic conditioning device of claim 12, wherein the control unit is adapted to determine a pre-set timing between a last respiration and an onset of the warning to the wearer, and to determine a pre-set timing between the warning to the wearer and onset of the painful pulse to the wearer.

14. A method of autonomic conditioning of a patient in treating sleep apnea comprising:
removably attaching a user mounting device to the patient, the user mounting device carrying at least one sensor and at least one actuator that are electrically connected to a control unit;
sensing a respiration cycle of the patient with the at least one sensor;
conditioning the patient to maintain a desired respiration cycle via the at least one actuator; and
negatively rewarding the patient for a failure to maintain the desired respiration cycle.

15. The method of claim 14, wherein conditioning the patient to maintain the desired respiration cycle includes warning the patient with a vibratory sensation delivered via the at least one actuator.

16. The method of claim 14, wherein negatively rewarding the patient for a failure to maintain the desired respiration cycle includes electrically shocking the patient via the at least one actuator.

17. The method of claim 14, wherein conditioning the patient to maintain the desired respiration cycle and negatively rewarding the patient for a failure to maintain the desired respiration cycle trains a patient's natural respiratory pacemaker to regain a normal functional control.
